(19) United States
(12) Patent Application Publication
(10) Pub. No.: US 2019/0175003 A1
(43) Pub. Date: Jun. 13, 2019

(54) IMAGE PICKUP UNIT FOR ENDOSCOPE AND ENDOSCOPE

(71) Applicant: OLYMPUS CORPORATION, Tokyo (JP)

(72) Inventors: Kazuhiro YOSHIDA, Okaya-shi (JP); Kensuke SUGA, Kamiina-gun (JP)

(73) Assignee: OLYMPUS CORPORATION, Tokyo (JP)

(21) Appl. No.: 16/276,873

(22) Filed: Feb. 15, 2019

Related U.S. Application Data
(63) Continuation of application No. PCT/JP2016/081895, filed on Oct. 27, 2016.

(51) Int. Cl. A61B 1/05; A61B 1/00; A61B 1/045

(52) U.S. Cl. CPC A61B 1/051; A61B 1/0008; A61B 1/0661; A61B 1/00009; A61B 1/00163; A61B 1/045

(57) ABSTRACT

An image pickup unit for endoscope includes: an image pickup portion having a rectangular parallelepiped shape on which a plurality of semiconductor devices including a first semiconductor device and a second semiconductor device are stacked, the image pickup portion including an image pickup device, a device stack bonded to a rear surface of the image pickup device, and a reinforcing member formed by resin covering an outer peripheral surface of the device stack; and a signal cable connected to the image pickup portion, in which: the first semiconductor device on a rear end side is smaller than the second semiconductor device on an image pickup device side; and the reinforcing member has a thickness on the rear end side that is thicker than a thickness on the image pickup device side.

### CROSS REFERENCE TO RELATED APPLICATION

This application is a continuation application of PCT/JP2016/081895 filed on Oct. 27, 2016, the entire contents of which are incorporated herein by this reference.

### BACKGROUND OF THE INVENTION

### 1. Field of the Invention

An embodiment of the present invention relates to an image pickup unit for endoscope that acquires an image, and an endoscope having a rigid distal end portion on which the image pickup unit for endoscope is arranged.

### 2. Description of the Related Art

An endoscope acquires an image of an inside of a body of a patient, for example, by inserting an insertion portion having a rigid distal end portion on which an image pickup unit is arranged into the inside of the body. Japanese Patent Application Laid-Open Publication No. 2005-334509 discloses an image pickup unit including an image pickup device in which a light receiving portion is formed, and a wiring board on which electronic components such as a capacitor, a resistor, and an IC chip configuring a drive circuit of the image pickup device are mounted. The wiring board is bonded on a rear surface of the image pickup device.

In the abovementioned image pickup unit, chip-shaped electronic components such as the capacitor, the resistor, and a buffer are mounted on the wiring board bonded to the rear surface of the image pickup device. Therefore, a length of the image pickup unit in an optical axis direction is long.

In recent years, semiconductor devices in which a planar device (thin film device) having functions of electronic components such as a capacitor is formed have been developed. The image pickup unit can be caused to be short and small by bonding a device stack obtained by stacking a plurality of semiconductor devices to a rear surface of the image pickup device. An endoscope having a short and small image pickup unit has a distal end rigid portion that is short in length, and hence is minimally invasive.

### SUMMARY OF THE INVENTION

According to an embodiment of the present invention, there is provided an image pickup unit for endoscope including: an image pickup portion including an image pickup device, a device stack, and a reinforcing member; and a signal cable connected to the image pickup portion, in which: the image pickup device includes a light receiving surface, and a rear surface opposite to the light receiving surface; the device stack bonded to the rear surface of the image pickup device is obtained by stacking a plurality of semiconductor devices including a first semiconductor device and a second semiconductor device; at least a part of an outer peripheral surface of the device stack is covered with the reinforcing member formed by resin; the first semiconductor device closest to a rear end side is smaller than the second semiconductor device on an image pickup device side; and the reinforcing member has a thickness on the rear end side that is thicker than a thickness on the image pickup device side.

An endoscope of another embodiment includes an image pickup unit for endoscope including: an image pickup portion including an image pickup device, a device stack, and a reinforcing member; and a signal cable connected to the image pickup portion, in which: the image pickup device includes a light receiving surface, and a rear surface opposite to the light receiving surface; the device stack bonded to the rear surface of the image pickup device is obtained by stacking a plurality of semiconductor devices including a first semiconductor device and a second semiconductor device; at least a part of an outer peripheral surface of the device stack is covered with the reinforcing member formed by resin; the first semiconductor device closest to a rear end side is smaller than the second semiconductor device on an image pickup device side; and the reinforcing member has a thickness on the rear end side that is thicker than a thickness on the image pickup device side.

### BRIEF DESCRIPTION OF THE DRAWINGS

FIG. 1 is a configuration diagram of an endoscope system including an endoscope of an embodiment;

FIG. 2 is a cross-sectional view of a distal end portion of the endoscope of the embodiment;

FIG. 3 is a cross-sectional view of an image pickup unit of the embodiment;

FIG. 4 is a top transparent view of the image pickup unit of the embodiment;

FIG. 5 is an exploded view of a device stack of the image pickup unit of the embodiment;

FIG. 6 is a layout drawing of the device stack of the image pickup unit of the embodiment;

FIG. 7 is a cross-sectional view of an image pickup unit of Modification 1;

FIG. 8 is a layout drawing of a device stack of the image pickup unit of Modification 1;

FIG. 9 is a cross-sectional view of an image pickup unit of Modification 2;

FIG. 10 is an exploded view of a device stack of an image pickup unit of Modification 3;

FIG. 11 is a perspective view of the image pickup unit of Modification 3;

FIG. 12 is a cross-sectional view of an image pickup unit of Modification 4; and

FIG. 13 is a cross-sectional view of an image pickup unit of Modification 5.

### DETAILED DESCRIPTION OF THE PREFERRED EMBODIMENT(S)

<Configuration of Endoscope System>

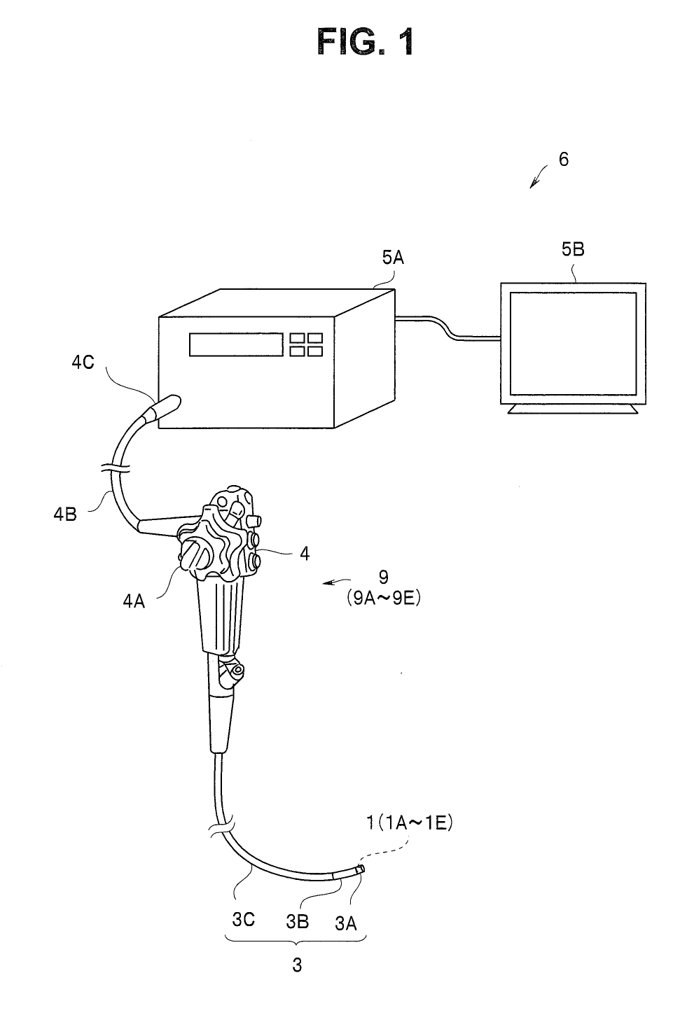

FIG. 1 illustrates an endoscope system 6 including an endoscope 9 of an embodiment. An image pickup unit 1 for endoscope of the embodiment (hereinafter also referred to as an “image pickup unit 1”) is arranged on a rigid distal end portion 3A of an insertion portion 3 of the endoscope 9.

Note that in the following description, the drawings based on each embodiment are schematic, and a relationship between a thickness and a width of each portion, a thickness ratio and a relative angle of each portion, and the like are different from actual relationships, thickness ratios, relative angles, and the like. Portions having different size relationships and ratios may also be included throughout the drawings. Further, drawing of some components may be omitted.

The endoscope 9 includes the insertion portion 3, a grasping portion 4 arranged on a proximal end portion side of the insertion portion 3, a universal cord 4B provided so as to extend from the grasping portion 4, and a connector 4C arranged on a proximal end portion side of the universal cord 4B. The insertion portion 3 includes the rigid distal end portion 3A on which the image pickup unit 1 is arranged, a bending portion 3B which is provided so as to extend on a proximal end side of the rigid distal end portion 3A, bendable, and for changing a direction of the rigid distal end portion 3A, and a flexible portion 3C provided so as to extend on a proximal end side of the bending portion 3B. An angle knob 4A that rotates and is an operation portion for an operator to operate the bending portion 3B is arranged on the grasping portion 4.

The universal cord 4B is connected to a processor 5A via a connector 4C. The processor 5A controls the entire endoscope system 6. In addition, the processor 5A performs signal processing on an image pickup signal outputted from the image pickup unit 1, and outputs the processed image pickup signal as an image signal. A monitor 5B displays the image signal outputted from the processor 5A as an endoscope image.

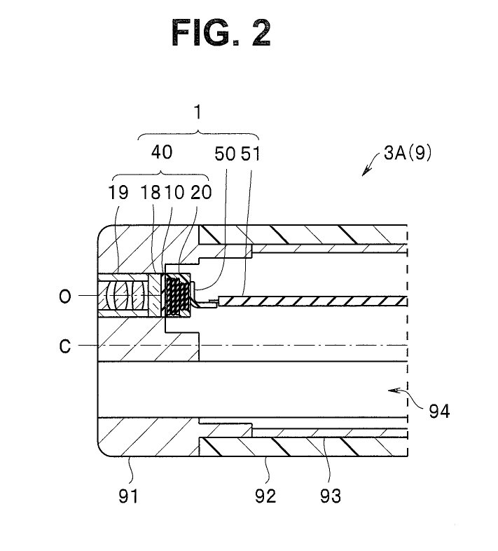

As illustrated in FIG. 2, in the rigid distal end portion 3A of the endoscope 9, an air feeding and water feeding tube 94, a cladding tube 92, an operation wire 93 connected to the angle knob 4A, a lens unit 19, and the image pickup unit 1 are arranged on a rigid distal end member 91. Note that the image pickup unit 1 is placed in a position in which an optical axis O is eccentric with respect to a central axis C of the rigid distal end portion 3A.

The lens unit 19 that fotins an object image includes a plurality of lenses and a lens holder. The lens unit 19 is inserted and fixed in a hole in the distal end member 91.

<Configuration of Image Pickup Unit>

Hereinafter, in the optical axis direction, a direction (Z-axis value increasing direction) in which an image pickup portion 40 is placed is referred to as a front side, and a direction (Z-axis value decreasing direction) in which a signal cable 51 is placed is referred to as a rear side. Further, in the direction orthogonal to the optical axis, a Y-axis direction is referred to as an upper direction/a lower direction, and an X-axis direction orthogonal to the Y-axis is referred to as a left direction/a right direction.

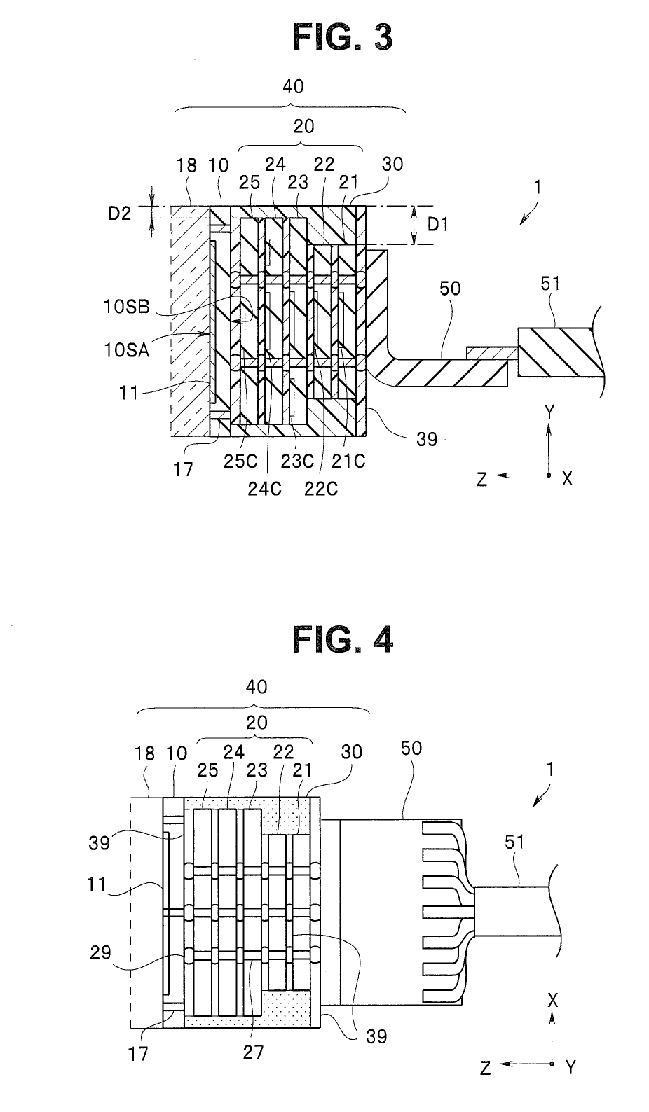

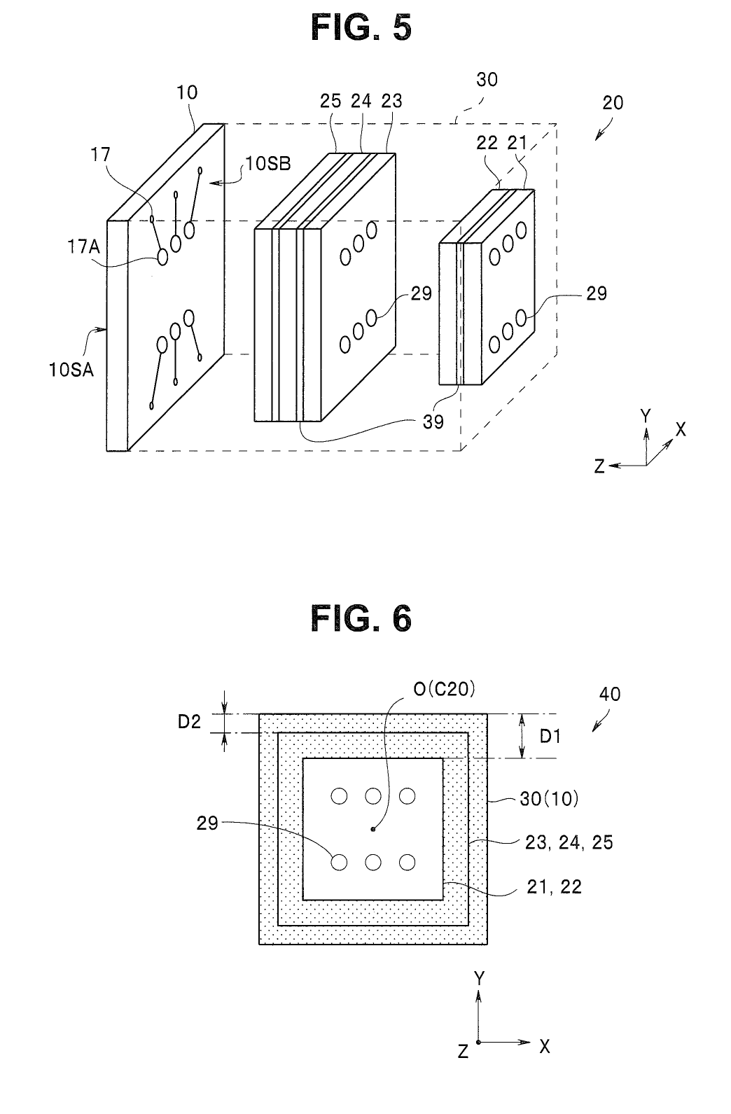

For example, as illustrated in FIG. 3 to FIG. 6, the image pickup unit 1 includes the image pickup portion 40 that is a substantially rectangular parallelepiped, a flexible wiring board 50 bonded to a rear end surface of the image pickup portion 40, and the signal cable 51. The signal cable 51 bonded to the wiring board 50 is connected to the universal cord 4B, and transmits an image pickup signal and the like.

Note that the signal cable 51 may be directly bonded to the image pickup portion 40. In other words, the wiring board is not a mandatory component of the image pickup unit 1.

The image pickup portion 40 includes the image pickup device 10 to which a cover glass 18 adheres, a device stack 20, and a reinforcing member 30 covering an entire surface of an outer peripheral surface of the device stack 20.

The image pickup device 10 having a rectangular shape in planar view, that is, the image pickup device 10 having a rectangular cross section in a direction orthogonal to the optical axis O includes a light receiving surface 10SA, a rear surface 10SB opposite to the light receiving surface 10SA, and four side surfaces. A light receiving portion 11 that receives the object image formed by the lens unit 19 and converts the received object image to an electrical signal is formed on the light receiving surface 10SA. The light receiving portion 11 is a CCD, a CMOS light receiving element, or the like, and generates an electrical signal by receiving light and performing photoelectric conversion. The light receiving portion 11 is connected to an electrode 17A on the rear surface 10SB via through wiring 17.

In the device stack 20, five semiconductor devices 21 to 25 including the first semiconductor device 21 and the second semiconductor device 23 are stacked. Each of the semiconductor devices 21 to 25 has a rectangular shape in planar view and is stacked via a sealing resin (underfilling) 39. The sealing resin 39 is an epoxy resin, an acrylic resin, a polyimide resin, a silicone resin, a polyvinyl resin, or the like.

The device stack 20 processes the electrical signal outputted from the image pickup device 10, and outputs the processed electrical signal as an image pickup signal. Planar devices 21C to 25C are formed on the semiconductor devices 21 to 25 each having a rectangular shape in planar view. Each of the planar devices 21C to 25C configures an electronic component function circuit such as a capacitor, a resistor, or a buffer, or a processing circuit such as a noise reduction circuit or an analog-to-digital converter circuit.

The plurality of semiconductor devices 21 to 25 may have different thicknesses. The planar devices 21C to 25C may be formed on one surface or both surfaces of the semiconductor devices 21 to 25, respectively. The number of the stacked semiconductor devices in the device stack 20 only needs to be two or more, and is not limited to five as in this example.

The semiconductor devices 21 to 25 are connected via respective through wiring 27 and bumps 29. A front end surface of the device stack 20 is connected to the electrode 17A on the rear surface 10SB of the image pickup device 10 via the bumps 29. A rear end surface of the device stack 20, that is, a rear surface of the semiconductor device 21 that is a rear end surface of the image pickup portion 40 is connected to the wiring board 50 via the bumps 29.

The sealing resin 39 is also filled between the rear surface 10SB of the image pickup device 10 and the front end surface of the device stack 20, and between the rear end surface of the device stack 20 and the wiring board 50, respectively.

External dimensions (sizes in planar view) of the semiconductor devices 21 to 25 in the direction orthogonal to the optical axis are equal to or less than a size of the image pickup device 10 in planar view. Therefore, the semiconductor devices 21 to 25 projected on a projection plane in the direction orthogonal to the optical axis are placed within a projection plane of the image pickup device 10.

The four side surfaces forming the outer peripheral surface of the device stack 20 is covered with the reinforcing member 30 formed by resin. In other words, the device stack 20 is embedded in the reinforcing member 30, an external form of which is formed by a rectangular parallelepiped-shaped hard material, and which hardly deforms even when applied with stress. A size of the reinforcing member 30 in planar view is substantially the same as a size of the image pickup device 10 in planar view.

The image pickup unit 1 including the device stack 20 is short and small. In addition, a size of the image pickup unit 1 in planar view is the same as the size of the image pickup device 10. Therefore, the image pickup unit 1 has a small diameter.

The device stack 20 is covered with the reinforcing member 30 formed by an epoxy resin, a fluorine resin, and the like that are hard resins having a Rockwell hardness on R scale (JIS K7202-2, a measured temperature is 23° C.) that is HR 100 or more, for example.

The periphery of the device stack 20 is reinforced by the reinforcing member 30 that is harder than the sealing resin 39, and hence the strength of the device stack 20 is enhanced. Note that the reinforcing member 30 also has better humidity resistance (water vapor barrier properties) than the sealing resin 39.

Note that at least one of the front end surface and the rear end surface of the device stack 20 besides the bumps 29 may be covered with the reinforcing member 30. In other words, the sealing resin 39 on the front end surface and the rear end surface of the device stack 20 may be the reinforcing member 30.

In the image pickup unit 1, an external dimension (size in planar view) of the first semiconductor device 21, which is arranged on a rear-most end side (proximal end portion side: a position closest to the signal cable 51) to which the wiring board 50 is bonded, in the direction orthogonal to the optical axis is smaller than a size of the second semiconductor device 23 arranged on the front side (image pickup device side: a position close to the image pickup device 10 with respect to the first semiconductor device 21) in planar view.

Therefore, the reinforcing member 30 covering the outer peripheral surface (four side surfaces) of the device stack 20 has a thickness D1 on the rear end side (proximal end portion side) that is thicker than a thickness D2 on the front side (image pickup device side). Note that the thicknesses D1 and D2 are lengths from the side surfaces of the respective semiconductor devices to an outer peripheral surface of the reinforcing member 30.

A stress in the shear direction applied to the image pickup unit 1 is larger on the rear end side than on the front end side. In the image pickup unit 1, the thickness D1 of the reinforcing member 30 on the rear end side to which a larger stress is applied is thicker than the thickness D2 on the front end side. Therefore, the image pickup unit 1 has a higher reliability than an image pickup unit in which the thickness of the reinforcing member 30 is uniform. The endoscope 1 including the image pickup unit 1 is highly reliable. The endoscope 1 includes the image pickup unit 1 that is short and small, and hence has a short rigid distal end portion and is minimally invasive.

Note that the strength improvement effect is notable if the thickness D1 is 120% or more of the thickness D2, but it is preferred that the thickness D1 be 150% or more of the thickness D2.

Note that in the image pickup unit 1, the semiconductor device 22 on the front side with respect to the first semiconductor device 21 is also the same size as the first semiconductor device 21. However, the semiconductor device 22 on the front side with respect to the first semiconductor device 21 may be larger than the first semiconductor device 21. This is because the rear end of the device stack 20 is applied with the largest stress, and the thickness D1 of the reinforcing member 30 covering the rear end is important.

As illustrated in FIG. 6, in the image pickup portion 40 of the image pickup unit 1, the optical axis O and a central axis C20 of the device stack 20 substantially match. Therefore, the thickness of the reinforcing member 30 is the same for four directions, that is, the upper direction, the lower direction, the left direction, and the right direction.

### MODIFICATIONS OF EMBODIMENT

Image pickup units 1A to 1E for endoscope and endoscopes 9A to 9E of Modifications 1 to 5 are similar to the image pickup unit 1 for endoscope and the endoscope 9 of the embodiment and has the same effect. Therefore, components having the same functions are denoted by the same reference characters and descriptions of the same functions are omitted.

### Modification 1

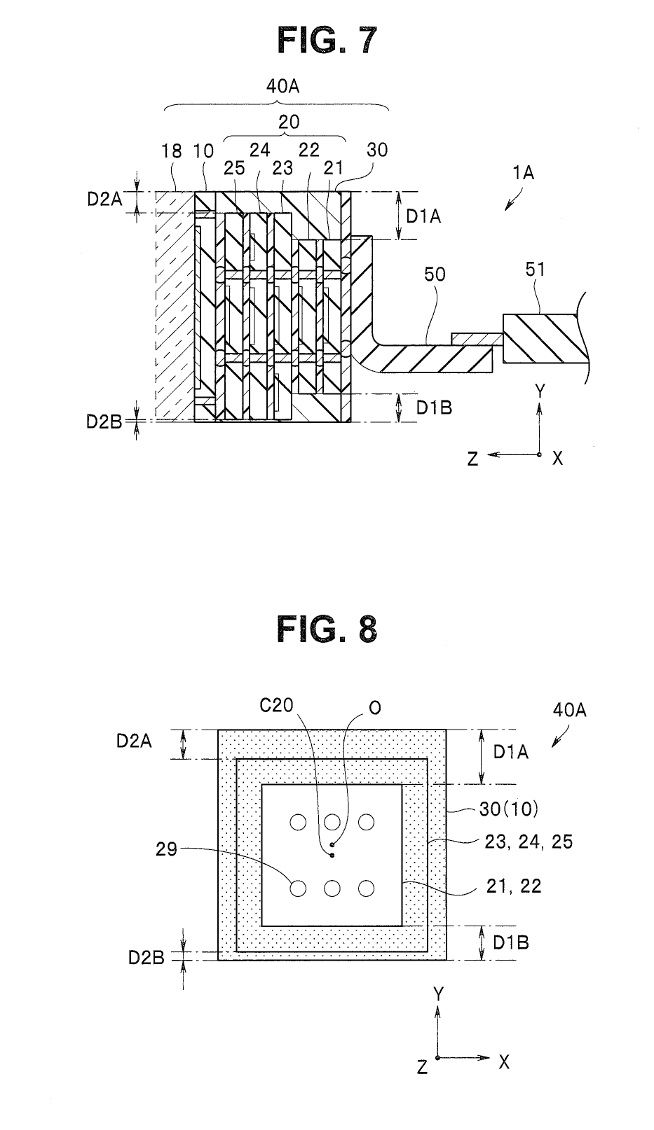

As illustrated in FIG. 7 and FIG. 8, in an image pickup unit 1A of Modification 1, thicknesses D1A and D2A on the outer peripheral side of the reinforcing member 30 are thicker than thicknesses D1B and D2B on the central axis side. In other words, as illustrated in FIG. 8, the central axis C20 of the device stack 20 is located on a lower side (central axis side) with respect to the optical axis O in an image pickup portion 40A of the image pickup unit 1A. Note that the thickness of the reinforcing member 30 is substantially the same in the left and right directions.

Although not shown, in an endoscope 9A, the optical axis O of the image pickup unit 1A is placed in a position eccentric with respect to the central axis C of the rigid distal end portion 3A as with the endoscope 9 that has been already described.

Therefore, the stress applied to the image pickup unit 1A is larger on the outer peripheral side of the rigid distal end portion 3A than on the central axis side. In the image pickup unit 1A, the reinforcing member 30 is thicker on the outer peripheral side (upper side) than on the central axis side (lower side). In the image pickup unit 1A, the outer peripheral portion to which large stress is applied has a stronger structure and a higher reliability as compared to the image pickup unit 1.

### Modification 2

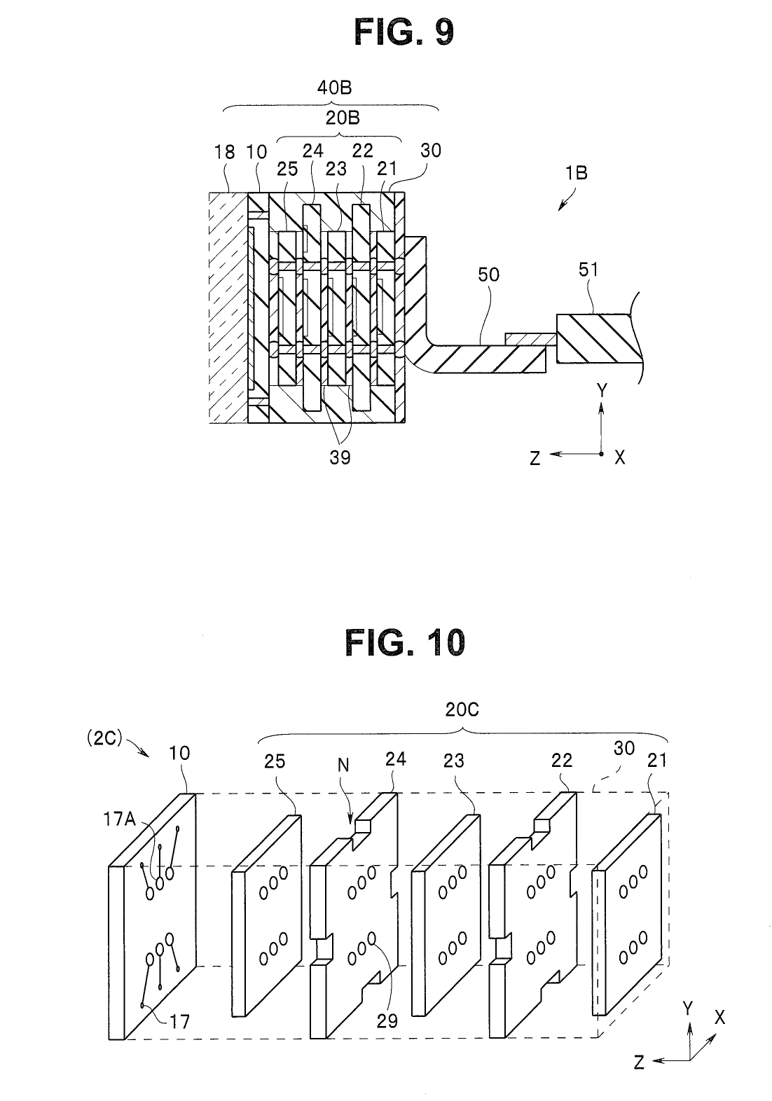

As illustrated in FIG. 9, in an image pickup unit 1B of Modification 2, the plurality of semiconductor devices 21 to 25 having different sizes are alternately stacked. In other words, the image pickup unit 1B includes the semiconductor devices 21 to 25 in a plurality of sizes, and the sizes of the semiconductor devices 21 to 25 stacked side-by-side are different from each other in a device stack 20B.

In other words, in the device stack 20B, the plurality of semiconductor devices 25 and 23 having the same size (external dimension in the direction orthogonal to the optical axis; size in planar view) as the first semiconductor device 21 on the rear end side (proximal end portion side) to which the wiring board 50 is bonded, and the semiconductor devices 22 and 24 larger than the first semiconductor device 21 are stacked in the order of the semiconductor devices 25, 24, 23, 22, and 21 from the front side.

In the image pickup unit 1B, the outer peripheral portions of main surfaces (front surfaces/rear surfaces) of the semiconductor device 22 and 24 in the device stack 20B are covered with the reinforcing member 30, and hence a contact area between the device stack 20B and the reinforcing member 30 is larger and the strength is enhanced as compared to the image pickup unit 1.

Further, as already described, the reinforcing member 30 has better humidity resistance (water vapor barrier properties) than the sealing resin 39 sealing spaces between the semiconductor devices 21 to 25. In the image pickup unit 1B, a distance from an outer surface of the reinforcing member 30 to side surfaces of the sealing resin 39 sealing each of the spaces between the semiconductor devices 21 to 25 is the same. Therefore, the humidity resistance is better than the humidity resistance of the image pickup unit 1.

### Modification 3

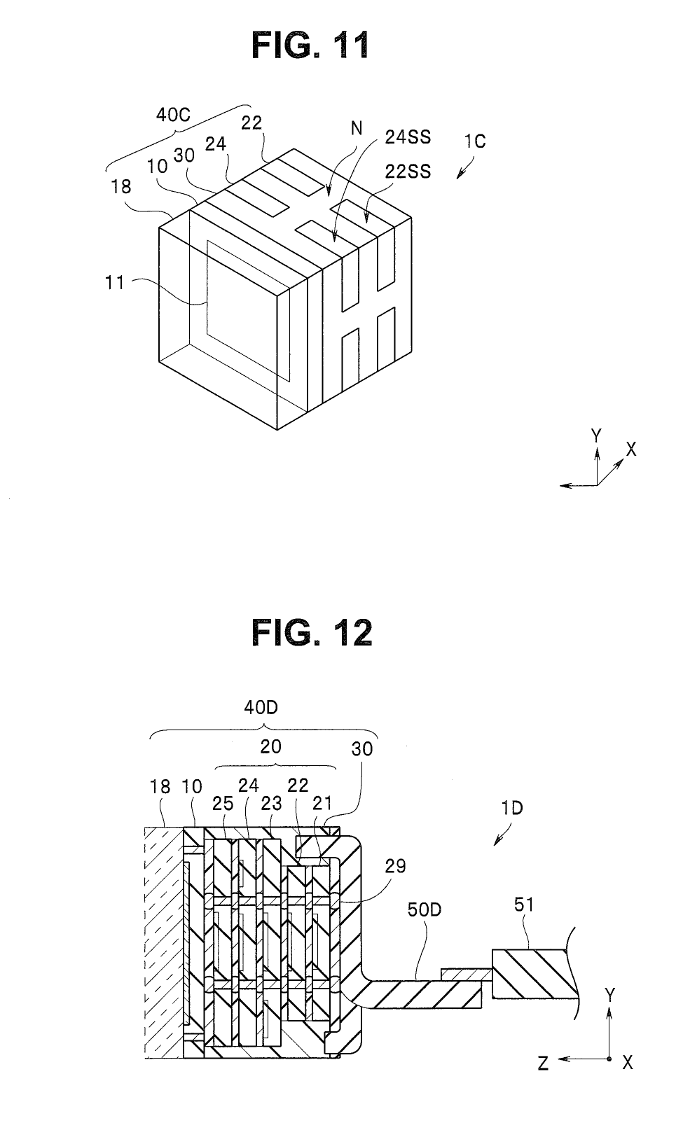

As illustrated in FIG. 10 and FIG. 11, an image pickup unit 1C of Modification 3 has cut-out portions N in the outer peripheral portions of the largest semiconductor devices 22 and 24, and a part of side surfaces 22SS, 24SS, and the like of the largest semiconductor devices 22 and 24 is exposed on the outer peripheral surface of a device stack 20C.

In other words, the size of the largest semiconductor devices 22 and 24 is the same as the size of the image pickup device 10. Therefore, a large planar device can be formed on the semiconductor devices 22 and 24. Further, the reinforcing member 30 of the image pickup unit 1C is not divided by the semiconductor devices 22 and 24 and is an integral structure due to the cut-out portions N.

Therefore, the image pickup unit 1C includes the large semiconductor devices 22 and 24 whose sizes are the same as the size of the image pickup device 10, but the strength is guaranteed.

Note that the plurality of semiconductor devices 21 to 25 having different sizes are alternately stacked in the image pickup unit 1C. However, the semiconductor devices do not necessarily need to be alternately stacked if cut-out portions are formed in the outer peripheral portion of the largest semiconductor device, and a part of a side surface of the largest semiconductor device is exposed on the outer peripheral surface of the device stack.

In the image pickup unit 1C, a part of an outer peripheral surface of a device stack 40C is not covered with the reinforcing member 30. In other words, only at least a part of the outer peripheral surface of the image pickup unit needs to be covered with the reinforcing resin 30.

### Modification 4

As illustrated in FIG. 12, in an image pickup unit 1D of Modification 4, slits are formed in a distal end portion of a flexible wiring board 50D in a direction parallel to the optical axis, and the distal end portion is divided into two. Further, the divided two distal end portions are embedded in the reinforcing member 30 above or below the device stack 20 of an image pickup portion 40D, respectively.

In the image pickup unit 1D, the stress applied to the wiring board 50D via the signal cable 51 is applied to the reinforcing member 30 via the distal end portions of the wiring board 50D. The image pickup unit 1D has a higher reliability because the image pickup unit 1D is reinforced by the wiring board 50D embedded in the reinforcing member 30 in a stronger manner.

Note that as already described, the reinforcing member 30 may also serve as the sealing resin 39 between the image pickup portion 40 and the wiring board 50, and the reinforcing member 30 may also cover the side surfaces of the wiring board 50.

### Modification 5

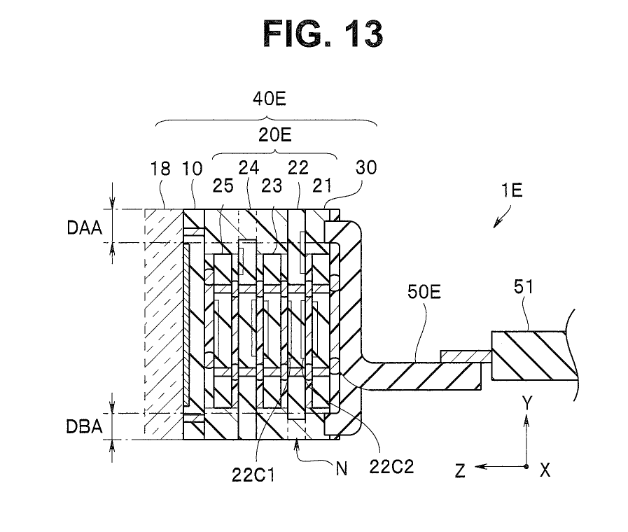

As illustrated in FIG. 13, an image pickup unit 1E of Modification 4 has four configurations of Modifications 1 to 4.

In other words, an average thickness DAA of the reinforcing member on an outer peripheral side (an upper side in the drawing) is thicker than an average thickness DBA of the rigid distal end portion 3A on a central axis C side (a lower side in the drawing), the plurality of semiconductor devices 21 to 25 having different sizes are alternately stacked, the cut-out portions N are formed in the outer peripheral portions of the largest semiconductor devices 22 and 24, a part of side surfaces of the largest semiconductor devices 22 and 24 is exposed on an outer peripheral surface of a device stack 20E, and distal end portions of a wiring board 50E are embedded in the reinforcing member 30.

The image pickup unit 1E has the effects of the image pickup units 1A to 1D of Modifications 1 to 4 in addition to the effect of the image pickup unit 1.

Note that needless to say, an image pickup unit including two or three configurations out of the configurations of the image pickup units 1A to 1D of Modifications 1 to 4 has the effects of the respective modifications.

Further, needless to say, the endoscopes 9A to 9E including the image pickup units 1A to 1E of Modifications 1 to 5 have the effect of the endoscope 9 and have the effects of the image pickup units 1A to 1E of Modifications 1 to 5. The endoscope of the present invention is not limited to a medical endoscope, but may be an industrial endoscope.

The present invention is not limited to the abovementioned embodiment and modifications, but various modifications, changes, and the like can be made within the range in which the gist of the present invention is not changed.

What is claimed is:

1. An image pickup unit for endoscope, comprising:
an image pickup portion comprising an image pickup device, a device stack, and a reinforcing member; and
a signal cable connected to the image pickup portion, wherein:
the image pickup device comprises a light receiving surface, and a rear surface opposite to the light receiving surface;
the device stack bonded to the rear surface of the image pickup device is obtained by stacking a plurality of semiconductor devices comprising a first semiconductor device and a second semiconductor device;
at least a part of an outer peripheral surface of the device stack is covered with the reinforcing member formed by resin;
the first semiconductor device closest to a rear end side is smaller than the second semiconductor device on an image pickup device side; and
the reinforcing member has a thickness on the rear end side that is thicker than a thickness on the image pickup device side.

2. The image pickup unit for endoscope according to claim 1, wherein:
the plurality of semiconductor devices comprise semiconductor devices in a plurality of sizes; and
sizes of the semiconductor devices stacked side-by-side in the device stack are different.

3. The image pickup unit for endoscope according to claim 1, wherein:
the plurality of semiconductor devices comprise semiconductor devices having different sizes;
a cut-out portion is formed in an outer peripheral portion of a largest semiconductor device out of the plurality of semiconductor devices; and
a part of a side surface of the largest semiconductor device is exposed on the outer peripheral surface of the device stack.

4. The image pickup unit for endoscope according to claim 1, comprising a flexible wiring board connecting the image pickup portion and the signal cable to each other, wherein the wiring board has a distal end portion embedded in the reinforcing member.

5. An endoscope, comprising
an insertion portion comprising a rigid distal end portion on which an image pickup unit for endoscope is arranged, and a bending portion provided so as to extend from the rigid distal end portion and configured to change a direction of the rigid distal end portion,
the image pickup unit for endoscope comprising:
an image pickup portion comprising an image pickup device, a device stack, and a reinforcing member; and
a signal cable connected to the image pickup portion, wherein:
the image pickup device comprises a light receiving surface, and a rear surface opposite to the light receiving surface;
the device stack bonded to the rear surface of the image pickup device is obtained by stacking a plurality of semiconductor devices comprising a first semiconductor device and a second semiconductor device;
at least a part of an outer peripheral surface of the device stack is covered with the reinforcing member formed by resin;
the first semiconductor device on a rear end side is smaller than the second semiconductor device on an image pickup device side; and
the reinforcing member has a thickness on the rear end side that is thicker than a thickness on the image pickup device side.

6. The endoscope according to claim 5, wherein:
the image pickup unit for endoscope is placed in a position eccentric with respect to a central axis of the rigid distal end portion; and
the reinforcing member has a thickness on an outer peripheral side that is thicker than a thickness on the central axis side.
